(19) United States
(12) Patent Application Publication
(10) Pub. No.: US 2023/0302075 A1
(43) Pub. Date: Sep. 28, 2023

(54) PREPARATIONS CONTAINING BERRY EXTRACTS FOR USE IN THE PROPHYLAXIS AND/OR TREATMENT OF VIRAL INFECTIONS CAUSED BY CORONAVIRIDAE

(71) Applicant: Evonik Operations GmbH, Essen (DE)

(72) Inventors: Jean-Luc HERBEAUX, Cascais (PT); Norbert WINDHAB, Hofheim (DE); Christoph BRÜCHER, Eschborn (DE); Anne BENEDIKT, Frankfurt (DE); Maria STEINKE, Hoechberg (DE); Jochen BODEM, Geroldshausen (DE)

(73) Assignee: Evonik Operations GmbH, Essen (DE)

(21) Appl. No.: 18/002,435

(22) PCT Filed: Jun. 18, 2021
(86) PCT No.: PCT/EP2021/066557

§ 371 Date: Dec. 19, 2022

(30) Foreign Application Priority Data
Jun. 26, 2020 (EP) 20182456.2

(51) Int. Cl. A61K 36/45; A61K 36/185; A61K 31/7048; A61K 9/00; A61P 31/14

(52) U.S. Cl. CPC A61K 36/45; A61K 9/0014; A61K 9/0078; A61K 31/7048; A61K 36/185; A61P 31/14; A61K 2236/53; A61K 2236/333

(57) ABSTRACT

Compositions may be used in treating or preventing a virus infection in a subject, wherein the virus is from the Coronaviridae family and wherein the composition includes an extract of black currants and/or bilberries. The black currants may be the fruit of Ribes nigrum, and/or the bilberries may be the fruit of Vaccinium myrtillus. The extract may be of the pomaces of the black currants and/or the bilberries.

The present invention is related to preparations containing one or more berry extracts for use in treating or preventing a virus infection in a subject, wherein the virus is from the Coronaviridae family and wherein the composition comprises an extract of black currants and/or bilberries.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, and apples and vegetables such as red cabbage. Bilberries, in particular Vaccinium myrtillus, and black currants, in particular Ribes nigrum, are especially suitable.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and — to a lesser extent — malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but delphinidin- and cyanidin-3-O-glucoside are also found (Gafner, Bilberry - Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

Coronaviridae is a large family of RNA viruses that cause infections which are connected to (severe) lung inflammations and diseases. In the last years several new virus types of the coronaviridae family like SARS and MERS caused huge numbers of infected individuals and high numbery of deaths. With the current corona pandemic caused by the new corona virus SARS-Cov2 and related to the lung disease Covid-19, the need for new methods for prevention and treatment against the virus is even more obvious and urgent than ever before.

In the context it was surprisingly found, that an extract of black currants and/or bilberries, can mediate inhibition of Coronaviridae virus infection and replication. Thus, the present invention is based on the use of Healthberry® 865 as anti-viral agent in the treatment and prophylaxis of Coronaviridae e.g. SARS-Cov2 infection. Therefore, extracts of black currants and/or bilberries could be an important solution for these respiratory infections by combining the antiviral effect with its positive influence on cell viability and no toxicity.

The present invention is related to a composition for use in treating or preventing a virus infection in a subject, wherein the virus is from the Coronaviridae family and wherein the composition comprises an extract of black currants and/or bilberries. In a preferred configuration the composition comprises both an extract of black currants and bilberries.

The composition according to the present invention is especially for use in treating or preventing a virus infection in human hosts selected from
Human coronavirus 229E
Human coronavirus NL63
Human coronavirus OC43
Middle East respiratory syndrome-related coronavirus (MERS-CoV),
Severe acute respiratory syndrome coronavirus (SARS-CoV),
Severe acute respiratory syndrome coronavirus 2 (SARS-CoV-2, COVID-19),
preferably SARS-CoV-2.

It is also preferred to use the composition according to the present invention for treating or preventing a virus infection with:
Bat coronavirus CDPHE15
Bat coronavirus HKU10
Rhinolophus ferrumequinum alphacoronavirus HuB-2013
Lucheng Rn rat coronavirus
Ferret coronavirus
Mink coronavirus 1
Miniopterus bat coronavirus 1
Miniopterus bat coronavirus HKU8
Myotis ricketti alphacoronavirus Sax-2011
Nyctalus velutinus alphacoronavirus SC-2013
Porcine epidemic diarrhea virus
Scotophilus bat coronavirus 512
Rhinolophus bat coronavirus HKU2
NL63-related bat coronavirus strain BtKYNL63-9b
China Rattus coronavirus HKU24
Human coronavirus HKU1
Murine coronavirus - type species
Bat Hp-betacoronavirus Zhejiang2013
Hedgehog coronavirus 1
Pipistrellus bat coronavirus HKU5
Tylonycteris bat coronavirus HKU4
Rousettus bat coronavirus GCCDC1
Rousettus bat coronavirus HKU9
Wigeon coronavirus HKU20
Bulbul coronavirus HKU11
Porcine coronavirus HKU15
Munia coronavirus HKU13
White-eye coronavirus HKU16
Night heron coronavirus HKU19
Common moorhen coronavirus HKU21
Beluga whale coronavirus SW1
Avian coronavirus

In a preferred embodiment, the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries.

It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%.

It is preferred, according to the present invention, when the extract is an alcoholic extract, preferably a methanol extract. The extract is preferably produced by a process comprising the steps of
extraction of black currants and/or bilberries,
purification via chromatography,
mixing of the extract(s) with water and
spray-drying of the mixture.

One example of such a process is disclosed in EP1443948.

In a preferred embodiment, maltodextrin is added to the composition.

The composition according to the present invention preferably contains at least three monosaccharide anthocyanins. Moreover, it preferably contains at least one monosaccharide anthocyanin in which the saccharide is arabinose or at least one disaccharide anthocyanin in which the disaccharide is rutinose. The composition preferably contains anthocyanins with at least two different aglycones, more preferably at least four. Especially preferably the composition contains anthocyanins in which the aglycone units are cyanidin, peonidin, delphinidin, petunidin, malvidin and optionally also pelargonidin. In one preferred embodiment, the composition also contains at least one trisaccharide anthocyanin. The disaccharide anthocyanins are more water soluble than the monosaccharides; moreover, cyanidin and delphinidin anthocyanins are amongst the most water soluble anthocyanins.

In an advantageous embodiment of the present invention anthocyanins are selected from cyanidin-3-glucoside, cyanidin-3-galactoside, cyanidin-3-arabinoside, delphinidin-3-glucoside, delphinidin-3-galactoside, delphinidin -3-arabinoside, petunidin-3- glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

The anthocyanins can be from natural sources or from synthetic productions. Natural sources are preferably selected from fruits, flowers, leaves, stems and roots, preferably violet petal, seed coat of black soybean. Preferably anthocyanins are extracted from fruits selected from: açaí, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin-3-rutinoside.

The counterion in the anthocyanins in the composition of the invention may be any physiologically tolerable counter anions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste. Besides the anthocyanins, the composition may desirably contain further beneficial or inactive ingredients, such as vitamins (preferably vitamin C), flavones, isoflavones, anticoagulants (e.g. maltodextrin, silica, etc.), desiccants, etc.

It is preferred when the composition comprises anthocyanins and is to be administered to the subject in a dose of the anthocyanins / regimen of 1 to 10 oral dosages of at least 80 mg anthocyanins each per day, preferably 3 to 6 oral dosages of at least 80 mg anthocyanins each per day.

In a further advantageous configuration, the composition is to be administered to the subject as parenteral bolus injection or infusion or parenteral nutritional solution. It is also preferred to use the composition to stabilize critical patients, where lifesaving treatments are not effective, and no last-line treatment is available (due to lack of treatment options).

The composition according to the present invention is to be administered to the subject, reaching a concentration in the target compartment at least 30 µg/ml, preferably at least 100 µg/ml. Target compartment are blood and lymph, specifically the medium surrounding the cells of the immune system, preferably Peripheral Blood Mononuclear Cells (PBMCs) or cells of the respiratory tract.

In a preferred embodiment, the subject is a human and preferably the subject is pregnant, or younger than 2 years or 65 years old or more.

The composition according to the present invention is preferably used when the subject suffers from asthma, chronic obstructive pulmonary disease or congestive heart failure.

A further aspect of the present invention is related to a liquid composition comprising an extract of black currants and/or bilberries, wherein the composition is comprised in a nebulizer, preferably a throat spray liquid composition comprising an extract of black currants and/or bilberries, wherein the composition further comprises an pharmaceutically acceptable excipient suitable for a liquid composition that is to be administered to the mucosal surfaces of the mouth and throat, preferably wherein the excipient comprises one or more of a tonicity adjusting agent, a buffering agent, a preservative, an antioxidant, a stabilizer, a pH adjusting agent, a penetration enhancer, a surfactant and a humectant.

The invention also provides a composition formulated as a throat spray, a throat lozenge or a mouthwash, further comprising a pharmaceutically acceptable carrier suitable for a composition that is to be administered to the mucosal surfaces of the mouth and throat.

The invention also provides a composition comprising an extract of black currants and/or bilberries, wherein the composition further comprises saline, and preferably wherein the composition is comprised in a nebulizer. The composition may comprise a tonicity adjusting agent, a buffering agent, a preservative, an antioxidant, a stabilizer, a pH adjusting agent, a penetration enhancer, a surfactant and a humectant. Preferably the composition is isotonic.

The present invention also refers to
a composition comprising an analgesic or an anti-inflammatory agent or topical anesthetic and an extract of black currants and/or bilberries, preferably wherein the analgesic is ibuprofen or paracetamol/acetaminophen,
a composition according for use in treating pain associated with a Coronaviridae infection in a subject,
a combined preparation comprising an analgesic or topical anesthetic, and an extract of black currants and/or bilberries, for simultaneous, separate or sequential use in medicine,
a throat lozenge comprising a topical anesthetic, and an extract of black currants and/or bilberries, preferably wherein the topical anesthetic is benzocaine or menthol,
a composition comprising an antipyretic, and an extract of black currants and/or bilberries,
a composition for use in treating fever associated with a Coronaviridae infection in a subject,
a combined preparation comprising an antipyretic, and an extract of black currants and/or bilberries, for simultaneous, separate or sequential use in medicine,
a combined preparation comprising palivizumab and an extract of black currants and/or bilberries for preventive treatment of Coronaviridae infection,
combination preparation comprising one or more of the following adrenaline, bronchodilators, steroids, antibiotics, ribavirin, salbutamol and an extract of black currants and/or bilberries for a treatment after Coronaviridae infection.

A combined preparation is one which comprises separately packaged active components which are to be combined in use, i.e. by being administered simultaneously, separately or sequentially to the subject.

Analgesic compounds are preferably selected from acetylsalicylic acid, Diclofenac, Dexibuprofen, Dexketoprofen, Flurbiprofen, Ibuprofen, Indometacin, Ketoprofen, Meloxicam, Nabumeton, Naproxen, Phenylbutazon, Piroxicam, Phenazon, Propyphenazon, rofecoxib, Celecoxib, Etoricoxib, Parecoxib, Metamizol, Paracetamol/Acetaminophen.

For all the compositions described above it is advantageous, when the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries. It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%. It is preferred, according to the present invention, when the extract is an alcoholic extract, preferably a methanol extract.

The present invention is also related to an antiviral agent for treating or preventing a virus infection in a subject, wherein the virus is from the Coronaviridae family with a level of efficacy of 1-2 log level, and an antiviral agent which is non-toxic.

The invention is also referring to an antiviral agent for treating or preventing a virus infection in a subject, wherein the virus is from the Coronaviridae family with a level of efficacy of 1-2 log level, which is not killing more than 30%, preferably not more than 20%, more preferably not more than 10% of cells in a cell-based assay in mammalian cells, preferably HepG2 cells.

This antiviral agent preferably comprises one or more anthocyanins selected from cyanidin-3-glucoside, cyanidin-3-galactoside, cyanidin-3-arabinoside, delphinidin-3-glucoside, delphinidin-3-galactoside, delphinidin -3-arabinoside, petunidin-3- glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

It is known that viral infections can occur when a medical device is used on a subject. This is particularly the case when the device, such as a catheter or feeding tube, is to be retained in the subject for any length of time, e.g. the dwell time of the device in the subject is more than 24 hours.

Accordingly, the present invention is also related to a composition for use, wherein the composition is for use with a medical device which is to be inserted into the subject, or wherein the subject has had a medical device inserted, optionally wherein the device is inserted via the nose or mouth. It is preferred, when the medical device is a needle, a catheter, a port, an intubation device or tube, or a nebulizer. It is further preferred, when a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.

The invention further refers to a medical device suitable for insertion into a subject, the medical device comprising a coating composition on an exterior surface of the device, wherein the coating composition comprising an extract of black currants and/or bilberries. It is preferred, when the medical device is a needle, a catheter, an intubation device or tube, or a nebulizer, preferably wherein the exterior surface of the medical device is plastic.

It is further preferred, when the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries. It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%. It is preferred, according to the present invention, when the extract is an alcoholic extract, preferably a methanol extract.

The invention also covers a method of making the medical device as described, the method comprising applying the coating composition to the exterior surface of the medical device, optionally wherein the coating composition is formulated as a cream, a hydrogel cream, or a spray.

Moreover, the invention refers to a deep-lung particle comprising a composition comprising an extract of black currants and/or bilberries, which is dispensed into the deeper respiratory tract of an individual and a device for dispensing a deep-lung particle into the deeper respiratory tract of an individual.

The composition may comprise a formulation of extracts of black currants and/or bilberries with nanoparticles, preferably liposomes. Such formulations may be inhaled to maximize the delivery of nanoparticles into the lung. Inhalation facilitates the localized delivery of compositions directly to the lungs via the oral or nasal inhalation route. For example, aerosolized delivery of liposomal interleukin-2 (IL-2) in dogs has been shown to be effective against pulmonary metastases from osteosarcoma (Khanna C, Anderson PM, Hasz DE, Katsanis E, Neville M, Klausner JS. Interleukin-2 liposome inhalation therapy is safe and effective for dogs with spontaneous pulmonary metastases. Cancer 1997; 79: 1409-21.) Moreover, the delivery of anticancer drugs via nanoparticles has been shown to be efficacious and safe in a variety of cancers. Anticancer drugs can also be formulated into drug nanocrystals with high drug loading and minimal use of excipients. (Sharad M, Wei G, Tonglei L, Qi Z, Review: Pulmonary delivery of nanoparticle chemotherapy for the treatment of lung cancers: challenges and opportunities, Acta Pharmacologica Sinica (2017) 38: 782-797).

In a preferred embodiment, a nanoparticle suspension comprising the composition according to the present invention is aerosolized into droplets with appropriate aerodynamic diameters using currently available inhalation devices. Such inhalation devices are preferably selected from nebulizers and pressurized metered dose inhalers (pMDI).

Therefore, in an advantageous configuration, the composition according to the present invention may also be formulated as nanoparticle suspension for use in a nebulizer. Such nebulizers convert suspension of nanoparticles into inhalable droplets and may be used for the delivery of the composition into the deep lungs without compromising liposome integrity. An alternative configuration refers to pMDIs, which create small inhalable droplets of drugs suspended in compressed propellant (such as hydrofluoroalkane (HFA)).

The present invention also refers to a nanoparticle formulation as a dry powder, which offers greater long-term stability than a suspension. Controlling the size of nanoparticles is central for their formulation into reliable and efficient inhalable dry powders. Nanoparticles can be dried with/without excipients via spray-drying, freeze-drying and spray freeze-drying to generate stable and uniformly sized inhalable particles.

In an alternative embodiment, nanoparticles may be co-dried with excipients, which leads to the formation of inhalable nanoparticle aggregates in an excipient matrix. It is possible to utilize particle engineering and ensure consistent and highly efficient delivery of nanoparticles to the lungs through nano-aggregates, large porous particles, and other formulation techniques.

Preferred embodiments for a composition for use in treating or preventing a virus infection in a subject of the present invention are summarized below:

In a preferred embodiment, the virus is from the Coronaviridae family and wherein the composition comprises an extract of black currants and/or bilberries.

In a preferred embodiment, the black currants are the fruit of Ribes nigrum and/or the bilberries are the fruit of Vaccinium myrtillus.

In a preferred embodiment, the composition contains an extract from black currants and bilberries in a weight ratio of 0.5:1 to 1:0.5.

In a preferred embodiment, the composition is an extract of the pomaces from black currants and bilberries.

In a preferred embodiment, the composition comprises anthocyanins and the anthocyanins are present in the composition at a concentration of at least 25 weight-%.

In a preferred embodiment, the extract is an alcoholic extract, preferably a methanol extract.

In a preferred embodiment, the extract is prepared by a process comprising the steps of extraction of black currants and bilberries, purification via chromatography, mixing of the extract(s) with water and spray-drying of the mixture.

In a preferred embodiment, the virus is from the Coronaviridae family and wherein the composition comprises one or more of the following anthocyanins:

cyanidin-3-glucoside, cyanidin-3-galactoside, cyanidin-3-arabinoside, delphinidin-3-glucoside, delphinidin-3-galactoside, delphinidin -3-arabinoside, petunidin-3- glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside, preferably one or more of cyanidin-3-glucoside, cyanidin-3-rutinoside, delphinidin-3-glucoside, delphinidin-3-rutinoside, cyanidin-3-galactoside and delphinidin-3-galactoside.

for use in treating or preventing a virus infection in a subject the virus is from the Coronaviridae family and wherein the composition comprises:

cyanidin-3-glucoside, cyanidin-3-rutinoside, delphinidin-3-glucoside, delphinidin-3-rutinoside, cyanidin-3-galactoside and delphinidin-3-galactoside.

In a preferred embodiment, the virus is selected from
Human coronavirus 229E,
Human coronavirus NL63,
Human coronavirus OC43,
Middle East respiratory syndrome-related coronavirus (MERS-CoV),
Severe acute respiratory syndrome coronavirus (SARS-CoV),
Severe acute respiratory syndrome coronavirus 2 (SARS-CoV-2, COVID-19), preferably SARS-CoV-2.

In a preferred embodiment, the virus is from the genus Betacoronavirus, preferably from the subgenus Sarbecovirus, more preferably from the species Severe acute respiratory syndrome-related coronavirus.

In a preferred embodiment, the virus is SARS-CoV-2.

In a preferred embodiment, the composition inhibits viral replication.

In a preferred embodiment, the composition comprises anthocyanins and is to be administered to the subject 1 to 10 oral dosages of at least 80 mg anthocyanins each per day, preferably 3 to 6 oral dosages of at least 80 mg anthocyanins each per day.

In a preferred embodiment, the composition is to be administered to the subject as parenteral bolus injection or infusion or parenteral nutritional solution to stabilize critical patients.

In a preferred embodiment, the composition is to be administered to the subject, reaching a concentration in the target compartment of at least 30 µg/ml, preferably at least 100 µg/ml.

In a preferred embodiment, the subject is a human.

In a preferred embodiment, the subject is human and is younger than 2 years old or is 65 years old or more.

In a preferred embodiment, the subject suffers from asthma, chronic obstructive pulmonary disease or congestive heart failure.

In a preferred embodiment, the composition is for use with a medical device which is to be inserted into the subject, or wherein the subject has had a medical device inserted, optionally wherein the device is inserted via the nose or mouth.

In a preferred embodiment, the medical device is a needle, a catheter, a port, an intubation device or tube, or a nebulizer.

In a preferred embodiment, a dwell time of the medical device in the subject is more than 24 hours, more than 48 hours, more than 72 hours, more than one week, more than 2 weeks, more than 3 weeks, preferably wherein the dwell time is more than one week, more than 2 weeks or more than 3 weeks.

Another preferred embodiment is directed to a liquid composition comprising an extract of black currants and/or bilberries, wherein the composition is comprised in a nebulizer.

Another preferred embodiment is directed to a throat spray liquid composition comprising an extract of black currants and/or bilberries, wherein the composition further comprises an pharmaceutically acceptable excipient suitable for a liquid composition that is to be administered to the mucosal surfaces of the mouth and throat, preferably wherein the excipient comprises one or more of a tonicity adjusting agent, a buffering agent, a preservative, an antioxidant, a stabilizer, a pH adjusting agent, a penetration enhancer, a surfactant and a humectant.

Another preferred embodiment is directed to a composition comprising an analgesic or topical anesthetic or anti-inflammatory agent, and an extract of black currants and/or bilberries, preferably wherein the analgesic is ibuprofen or paracetamol/acetaminophen.

Another preferred embodiment is directed to a composition according to the present invention for use in treating pain associated with a Coronaviridae infection in a subject.

Another preferred embodiment is directed to a combined preparation comprising an analgesic or topical anesthetic, and an extract of black currants and/or bilberries, for simultaneous, separate or sequential use in medicine.

Another preferred embodiment is directed to a throat lozenge comprising a topical anaesthetic, and an extract of black currants and/or bilberries, preferably wherein the topical anesthetic is benzocaine or menthol.

Another preferred embodiment is directed to a composition comprising an antipyretic, and an extract of black currants and/or bilberries.

Another preferred embodiment is directed to a composition according to the present invention for use in treating fever associated with a Coronaviridae infection in a subject.

Another preferred embodiment is directed to a combined preparation comprising an antipyretic, and an extract of black currants and/or bilberries, for simultaneous, separate or sequential use in medicine.

Another preferred embodiment is directed to a medical device suitable for insertion into a subject, the medical device comprising a coating composition on an exterior surface of the device, wherein the coating composition comprising an extract of black currants and/or bilberries.

Another preferred embodiment is directed to a medical device, wherein the medical device is a needle, a catheter, an intubation device or tube, or a nebulizer, preferably wherein the exterior surface of the medical device is plastic.

In a preferred embodiment, the method comprising applying the coating composition to the exterior surface of the medical device, optionally wherein the coating composition is formulated as a cream, a hydrogel cream, or a spray.

Another preferred embodiment is directed to a method for treating or preventing a virus infection in a subject in need thereof, comprising administering to the subject an effective amount of a composition comprising an extract of black currants and/or bilberries, wherein the virus is from the Coronaviridae family.

Another preferred embodiment is directed to a method for preventing a device-associated virus infection in a subject, comprising: (a) inserting a device into the subject and administering an effective amount of a composition comprising an extract of black currants and/or bilberries at a site of insertion of the device; and/or (b) applying an effective amount of a composition comprising an extract of blackcurrants and bilberries to an external surface of a device and inserting the device into the subject, wherein the virus is from the Coronaviridae family.

In a preferred embodiment, the extract is as defined above.

In a preferred embodiment, the virus is as defined above.

In a preferred embodiment, the composition is to be administered as defined above.

### EXAMPLES

The berry extracts composition (Healthberry® 865; Evonik Nutrition & Care GmbH, Darmstadt, Germany) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The relative content of each anthocyanin in the Healthberry® 865 product was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin; 2.5% of 3-O-b-glucosides, 3-O-b-galactosides, and 3-O-b-arabinosides of petunidin; 2.5% of 3-O-b-glucosides, 3-O-b-galactosides, and 3-O-b-arabinosides of peonidin; 3.0% of 3-O-b-glucosides, 3-O-b-galactosides, and 3-O-b-arabinosides of malvidin.

The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around 40 weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

### Materials

TABLE 1

Materials used for the measurement of cell survival and metabolism

| | |
|---|---|
| Material | Supplier |
| RealTime-Glo™ MT Cell Viability Assay | Promega GmbH, Mannheim (Germany) |
| Dulbecco’s Modified Eagle’s medium (DMEM) | Gibco Life technologies, Carlsbad (USA) |
| Fetal bovine serum | Gibco Life technologies, Carlsbad (USA) |
| Vero cells | ATCC/American Type Culture Collection in Partnership with LGC standards, Wesel (Germany) |
| Healthberry® 865 (anthocyanin content 29.7%) | Evonik Nutrition & Care GmbH, Darmstadt (Germany) |

TABLE 2

Devices used for the measurement of cell survival and metabolism

| | |
|---|---|
| Device | Supplier |
| Centro LB 960 microplate luminometer | Berthold Technologies, (Germany) |

TABLE 3

Materials used for anti-viral assay

| | |
|---|---|
| Material | Supplier |
| SARS-CoV 2, Coronavirus (COVID-19) | patient derived isolate (Institute of Virology Würzburg) |
| Influenza virus serotype A | patient derived isolate (Institute of Virology Würzburg) |
| Dulbecco’s Modified Eagle’s medium (DMEM) | Gibco Life technologies, Carlsbad (USA) |
| Fetal bovine serum | Gibco Life technologies, Carlsbad (USA) |
| Vero cells | ATCC/American Type Culture Collection in Partnership with LGC standards, Wesel (Germany) |
| MDCK cells | ATCC/American Type Culture Collection in Partnership with LGC standards, Wesel (Germany) |
| HP Viral Nucleic Acid Kit | Hoffman-La-Roche Ltd., Basel (Switzerland) |
| RTqPCR LightMix® Modular Influenza A kit (Cat. No. 07 792 182 001) | Hoffman-La-Roche Ltd., Basel (Switzerland) |
| LightCycler® Multiplex RNA Virus Master kit (Cat. No. 07 083 173 001) | Hoffman-La-Roche Ltd., Basel (Switzerland) |
| MagNA Pure 24 system | Hoffman-La-Roche Ltd., Basel (Switzerland) |
| TIB MOLBIOL LightMix Assay SARS-CoV-2 RdRP RTqPCR assay kit | Hoffman-La-Roche Ltd., Basel (Switzerland) |
| RNA Process Control PCR Kit | Hoffman-La-Roche Ltd., Basel (Switzerland) |
| Healthberry® 865 (anthocyanin content 29.7%) | Evonik Nutrition & Care GmbH, Darmstadt (Germany) |
| Bilberry extract, Vaccinium myrtillus (anthocyanin content 38.8%) | Evonik Nutrition & Care GmbH, Darmstadt (Germany) |
| Black currant extract, Ribes nigrum (anthocyanin content 30%) | Evonik Nutrition & Care GmbH, Darmstadt (Germany) |

TABLE 4

Devices used for the anti-viral assay

| | |
|---|---|
| Device | Supplier |
| LightCycler480 II qPCR machine | Hoffman-La-Roche Ltd., Basel (Switzerland) |
| LHS laboratory robot | BRAND GmbH & CO KG, Wertheim (Germany) |

### Methods

### Test Compound Preparation

All test compounds were dissolved and diluted in cell culture medium. The overall amount of anthocyanins was normalized between Healthberry® 865 and the single anthocyanins (e.g. 500 µg/mL of Healthberry® 865 corresponds to 150 µg/mL of anthocyanins tested for the single test compounds) or as well the single berry extracts (taken into account that Healthberry® 865 also contains maltodextrin besides the anthocyanins). The medium served as control for viral inhibition or cytotoxicity.

### Cell Viability Assay

Cell viability was measured by RealTime-Glo™ MT Cell Viability Assay (Cat. No. G9712, Promega, Germany). Vero cells (2×10⁴) were incubated with decreasing amounts of the compound solubilized in DMEM. Wells with DMEM alone served as control. The MT Cell Viability Substrate and the NanoLuc® luciferase were added according to the manufacturer’s instructions. The assays were performed in triplicates. After 1 h, and then every six or 12 h, the luminescence was measured with Centro LB 960 microplate luminometer (Berthold Technologies, Germany). Luminescence values after 1 h were set to 1 and changes over time were calculated.

### Anti-Viral Assay

### SARS-CoV 2 Viral Load Determination (RTqPCRs)

To analyze the influence of the test compounds on SARS-2 coronavirus replication, Vero cells were pre-incubated with Healthberry® 865, Bilberry extract or Black currant extract at concentrations of 500, 250 and 125 µg/mL. The cells were infected with a patient derived SARS-2 Coronavirus (COVID-19). Cellular supernatants were collected 3 days after infection and centrifuged at 2000 rpm for 5 min to remove detached cells. Viral RNAs were extracted using a MagNA Pure 24 system (Roche, Germany). SARS-CoV-2 RNA genomes were quantified with the TIB MOLBIOL LightMix Assay SARS-CoV-2 RdRP RTqPCR assay kit with RNA Process Control PCR Kit (Roche). The PCR reaction setup was performed using a BRAND LHS laboratory robot to ensure quality. The amplification was performed with a LightCycler 480 II (Roche).

From the first identification till now, antiviral compounds are initially identified via screening assay either in vitro or in cell culture using replication assays. Even the activities of compounds identified by in vitro enzyme screening tests need to be verified in cell culture-based assays. These assays are state of the art methods to identify and confirm antiviral activities since they allow the quantification of the inhibition of viral replication and ensure the cellular uptake of compounds. For example, aciclovir, the gold standard in the treatment of HSV-1, was identified by screening of antiviral substances in sponges (Elion et al., 1977 Selectivity of action of an antiherpetic agent, 9-(2-hydroxyethoxymethyl)guanine. PNAS 74. 5716). Later, the antiviral activity of aciclovir inhibiting other members of the Herpesviridae was shown in cell culture-based assays as well (AKESSON-JOHANSSON et al., 1990 Inhibition of Human Herpesvirus 6 Replicationby9-[4-Hydroxy-2-(Hydroxymethyl)Butyl]Guanine (2HM-HBG) and Other Antiviral Compounds. AAC 34. 2417). Moreover, all compounds used as clinical drugs against HIV-1, such as 3TC and Lopinavir (ABT-378), were initially tested in vitro to demonstrate their antiviral effects (Coates et al., 1992. The Separated Enantiomers of 2′-Deoxy-3′-Thiacytidine (BCH 189) Both Inhibit Human Immunodeficiency Virus Replication In Vitro. AAC 36. 202; Sham et al. 1998. ABT-378, a Highly Potent Inhibitor of the Human Immunodeficiency Virus Protease. AAC 42. 3218).

### Influenza Genome Determination

MDCK cells were seeded in 48 well plates. After 24 h test compounds were added, and cells were subsequently infected with influenza A virus. All infections were performed in triplicates. Cell culture supernatants were harvested three days post-infection and centrifuged at 2000 rpm to remove detached cells and analyze viruses secreted to the supernatant. Viral RNAs were isolated from 200 µl cell culture supernatants using the Roche HP Viral Nucleic Acid Kit according to the manufacturer’s manual. Viral genome copy numbers were determined using 5 µl of the eluted RNA and the RTqPCR LightMix® Modular Influenza A kit (Cat. No. 07 792 182 001, Roche) in combination with the LightCycler® Multiplex RNA Virus Master kit (Cat. No. 07 083 173 001, Roche). All PCR reactions were performed in triplicates from a RNAs with a Roche LightCycler96 qPCR 20. The Cq values were determined with the respective cycler software (Roche Lighcylcler96 Application software V1.1). The internal standard of the Modular Influenza A kit with 1000 genome copies served as positive control. Quality was ensured by following the MIQE guidelines.

### Example 1: Influence of Berry Extracts on Cell Viability

To exclude cellular toxicity and adverse side effects, cellular viabilities of the test compounds on Vero cells (96 well-plate: 650 cells/well) were determined with the RealTime-Glo™ MT Cell Viability Assay kit. This assay measures the intracellular ATP content and therefore provides information on the cellular viability and metabolism. The cells were incubated with decreasing compound concentration in triplicate assays. Subsequently, both the MT Cell Viability Substrate and NanoLuc® Enzyme were added, and the luciferase activities were measured after 1 h. These measurements were repeated every 6 h or 12 h, and changes to the luciferase activity at the beginning of the experiment were calculated per individual well. The luminescence was normalized on the mean of the medium control wells for each time-point. These compensations result in values of 1 for the medium control at each time point. Values less than 1 indicate a lower number of cells or a decrease in metabolic activity compared to the appropriate controls.

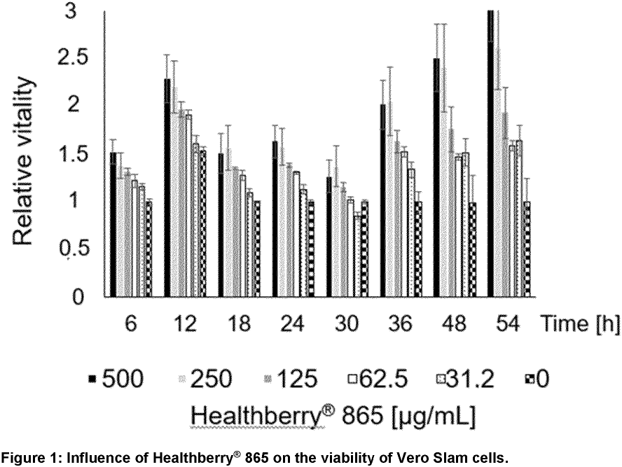

FIG. 1 displays the influence of Healthberry® 865 on the viability of Vero cells. The increase of luciferase activity, measured at nine different time-points, was normalized to the increase of control cells incubated with the medium. Error bars represent the standard deviation.

Healthberry® 865 did not negatively influence cellular growth or metabolic activity at any concentration analysed, indicating the compound was non-toxic at these concentrations. Healthberry® 865 even led to increased metabolic activity and viability of the cells when incubated with increasing concentrations of the extract.

### Example 2: Anti-Viral Effects of Berry Extracts on SARS-CoV-2 Virus

To analyze the influence of the test compounds on SARS-2 coronavirus replication, Vero cells were pre-incubated with Healthberry® 865, bilberry extract or black currant extract at concentrations of 500, 250 and 125 µg/mL. The cells were infected with a patient derived SARS-2 Coronavirus (COVID-19). Cellular supernatants were collected 3 days after infection and centrifuged at 2000 rpm for 5 min to remove detached cells. Viral RNAs were extracted using a MagNA Pure 24 system (Roche, Germany). SARS-CoV-2 RNA genomes were quantified with the TIB MOLBIOL LightMix Assay SARS-CoV-2 RdRP RTqPCR assay kit with RNA Process Control PCR Kit (Roche). The PCR reaction setup was performed using a BRAND LHS laboratory robot to ensure quality. The amplification was performed with a LightCycler 480 II (Roche).

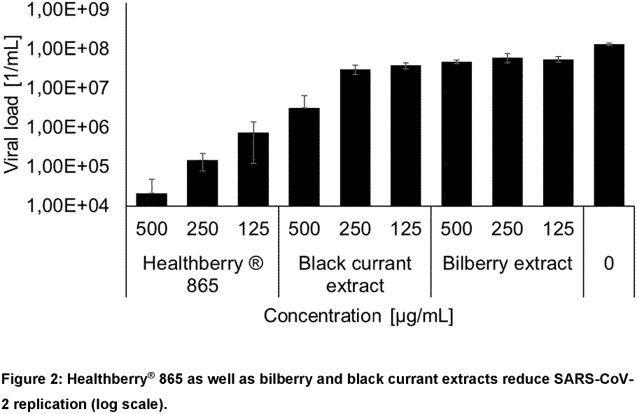

FIG. 2 shows that Healthberry® 865 as well as bilberry and black currant extracts reduce SARS-CoV-2 replication (log scale). Vero cells were treated with Healthberry® 865 as well as bilberry and black currant extracts and subsequently infected with SARS-CoV-2. Viral load was determined using RTqPCR. Error bars indicate the standard deviation of three independent samples.

Since Healthberry® 865 is a composition of bilberry and black currant extracts all three extracts were tested. The results of these anti-viral assays show, that Healthberry® 865 has a strong effect on SARS-CoV-2 replication even at lower concentrations, whereas the single berry extracts from black currant suppresses SARS-CoV-2 viral infection by approximately 1.5 orders of magnitude in a concentration of 500 µg/mL This indicates the potential use of these berry extracts and especially for mixtures of bilberry and black currant extracts, in treatment and prevention of SARS-CoV-2 infections and Covid-19 disease.

### Example 3: Anti-Viral Effects of Berry Extracts on Influenza A Virus (Comparative)

The influence of Healthberry® 865 and single anthocyanins on the replication of Influenza A virus were analyzed. MDCK cells were incubated with the test compounds and subsequently infected with a patient-derived isolate of Influenza virus serotype A. All reactions were performed in triplicates. Cell culture supernatants were harvested after three days, and viral genomic RNAs were isolated from 200 µl cell culture supernatants. Viral loads were determined by RTqPCR using the LightMix® Modular Influenza A kit (Roche). Positive controls with 1000 Influenza genome copies were included in the RTqPCR. All RTqPCR reactions were performed in triplicates.

All test materials, including Healthberry® 865, showed similar amounts of virus in the supernatant as the negative control, with only minor differences indicating that none of the components inhibited influenza virus replication.

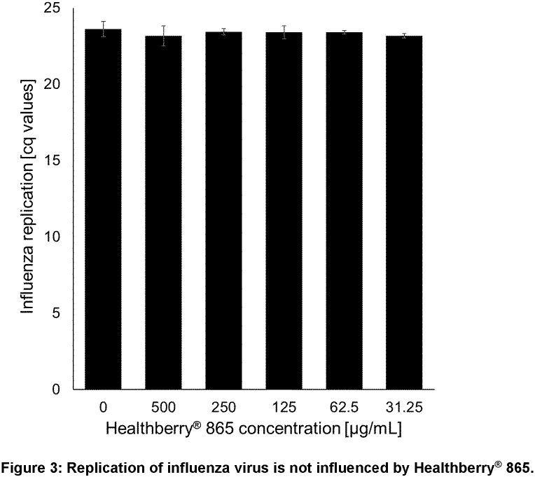

FIG. 3 shows that the replication of influenza virus is not influenced by Healthberry® 865. MDCK cells were pretreated with Healthberry® 865, infected with influenza virus (serotype A). Viral RNAs were isolated and quantified by RTqPCR (Cq-values; note: lower Cq values correspond to higher viral loads).

The results displayed no effect of Healthberry® 865 on Influenza A virus confirming the specificity of the anti-viral effects of berry extracts of black currants and bilberries on specific viruses or virus families, respectively. Other compounds as the single anthocyanins also did not show any influence on the replication of influenza virus.

1. A composition, comprising:
an extract of black currants and/or bilberries,
wherein the composition is suitable for use in treating or preventing a virus infection in a subject, and
wherein the virus infection is from a Coronaviridae family virus and wherein the composition.

2. The composition of claim 1, wherein the black currants are fruit of Ribes nigrum, and/or
wherein the bilberries are fruit of Vaccinium myrtillus.

3. The composition of claim 1, wherein the extract is of pomaces from the black currants and the bilberries.

4. The composition of claim 1, wherein the composition comprises anthocyanins, and
wherein the anthocyanins are present in the composition at a concentration of at least 25 wt.%.

5. The composition of claim 1, wherein the extract is an alcoholic extract.

6. The composition of claim 1, wherein the extract is prepared by a process comprising:
extracting the black currants and the bilberries, to obtain an extract;
purifying the extract via chromatography, to obtain a purified extract,
mixing the purified extracts with water, to obtain a mixture; and
spray-drying the mixture.

7. The composition of claim 1, wherein the composition comprises:
cyanidin-3-glucoside, cyanidin-3-galactoside, cyanidin-3-arabinoside, delphinidin-3-glucoside, delphinidin-3-galactoside, delphinidin -3-arabinoside, petunidin-3- glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside, or a mixture thereof.

8. The composition of claim 1, wherein the composition comprises:
cyanidin-3-glucoside, cyanidin-3-rutinoside, delphinidin-3-glucoside, delphinidin-3-rutinose, cyanidin-3-galactoside, and delphinidin-3-galactoside.

9. The composition of claim 1, wherein the virus is
Human coronavirus 229E,
Human coronavirus NL63,
Human coronavirus OC43,
Middle East respiratory syndrome-related coronavirus (MERS-CoV),
Severe acute respiratory syndrome coronavirus (SARS-CoV), or
Severe acute respiratory syndrome coronavirus 2 (SARS-CoV-2, COVID-19).

10. The composition of claim 1, wherein the composition comprises anthocyanins and is configured to be administered to the subject in 1 to 10 oral dosages of at least 80 mg anthocyanins each per day.

11. The composition of claim 1, wherein the composition is configured to be administered to the subject, reaching a concentration in a target compartment of at least 30 µg/mL.

12. The composition µg/ml, wherein the subject is a human.

13. The composition µg/ml, wherein the subject suffers from asthma, chronic obstructive pulmonary disease, or congestive heart failure.

14. A nebulizer, comprising:
a liquid composition comprising an extract of black currants and/or bilberries,
wherein the liquid composition further comprises an pharmaceutically acceptable excipient suitable for the liquid composition to be administered to the mucosal surfaces of the mouth and throat.

15. A combined preparation, comprising:
an analgesic or topical anesthetic; and
an extract of black currants and/or bilberries,
wherein the combined preparation is configured for simultaneous, separate, or sequential use in medicine.

16. The composition of claim 1, wherein the extract is from the black currants and the bilberries and is in a weight ratio in a range of from 0.5:1 to 1:0.5.

17. The composition of claim 2, wherein the extract is from the black currants and the bilberries and is in a weight ratio in a range of from 0.5:1 to 1:0.5.

18. The composition of claim 1, wherein the extract is a methanol extract.

19. The composition of claim 1, wherein the virus is Severe acute respiratory syndrome coronavirus 2 (SARS-CoV-2, COVID-19).
